(19) United States
(12) Patent Application Publication
(10) Pub. No.: US 2024/0000543 A1
(43) Pub. Date: Jan. 4, 2024

(54) METHODS OF ENDONTIC THERAPY UTILIZING LASERS AND ANTIMICROBIAL MATERIALS

(71) Applicant: CAO Group, Inc., West Jordan, UT (US)

(72) Inventor: Steven D Jensen, South Jordan, UT (US)

(73) Assignee: CAO Group, Inc., West Jordan, UT (US)

(21) Appl. No.: 17/904,538

(22) PCT Filed: Feb. 26, 2021
(86) PCT No.: PCT/US21/20094

§ 371 Date: Sep. 1, 2022

Related U.S. Application Data
(60) Provisional application No. 62/982,607, filed on Feb. 27, 2020.

(51) Int. Cl. A61C 5/50; A61K 6/54; A61K 6/65; A61K 6/62; A61K 6/69

(52) U.S. Cl. CPC A61C 5/50; A61K 6/54; A61K 6/65; A61K 6/62; A61K 6/69

(57) ABSTRACT

An improved method of endodontic therapy utilizes two strategies to create long lasting and effective dental restoration. After a root canal is prepared by conventional means, a dye is flooded into the canal to stain residual infected tissue. The dye is selected to enhance the absorption of radiant energy by dyed tissue by matching at least one λₘₐₓ of the dye to the output of a laser. The radiant energy (24) from the laser is then used to obliterate all infected root tissues (16) remaining within the prepared root canal (14). Laser treatment extends from the root apex (12) to the tooth corona (18). Second, cements utilizing an anti-microbial agent such as zinc pyrithione are used to restore the tooth, giving the restoration inherent anti-microbial properties. While ideally used together, each strategy may also be utilized on its own for improvement over the prior art.

### CROSS-REFERENCES TO RELATED APPLICATIONS

The present invention claims priority to prior filed U.S. Application No. 62/982,607, filed on Feb. 27, 2020, and incorporates the same by reference herein in its entirety.

### FIELD OF THE INVENTION

The present invention relates to the field of dental treatment and more particularly relates to a method of endodontic therapy utilizing lasers and selected dyes that enhance absorption of radiant energy by tissues.

### BACKGROUND OF THE INVENTION

Conventional endodontic treatment usually comprises opening the tooth to gain access to the root. After it is exposed, necrotic root is extirpated with endodontic files, leaving an empty root canal. The exposed root canal is then irrigated with sodium hypochlorite for immediate disinfection. The root canal is then obturated with gutta percha. If necessary, a cap may then be positioned over a remaining stump of the tooth, sealing the work area from above. However, this does not address sealing the work from below the gingiva. It is critical that the root canal be totally sealed and not allow seepage beyond the apex of the tooth's root because this could result in a secondary infection from residual infected tissue that was never removed during the initial procedure. Conventional extirpation and disinfection methods are intended to treat most of the infected tissue, but they also tend to not address all infected tissue. Some infected tissue invariably is left behind. Root leakage, or other leakage from the pulp chamber, is the most significant cause of endodontic re-treatment. The present invention is a method utilizing a laser and dye selected to enhance the absorption of radiant energy by dyed tissues to obliterate all the necrotic root tissue. Zinc pyrithione also may be utilized in endodontic cement or sealer to add anti-microbial characteristics to the restoration.

### SUMMARY OF THE INVENTION

In view of the foregoing disadvantages inherent in the known types of endodontic treatment, an improved method may provide a more effective means to clean out necrotic tissue, add anti-microbial properties to the restoration, and enhance the stability and longevity of the final dental restoration. As such, a new and improved methods and materials may comprise utilizing a laser and matched dye to enhance the extirpation of necrotic tissue and adding and anti-microbial such as zinc pyrithione to restorative materials such as dental cements to accomplish these objectives.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective view, in partial section, showing laser obliteration of necrotic root tissue in a tooth.

FIG. 2 is a perspective view, in partial section, showing dental restoration of the tooth treated in FIG. 1.

### DETAILED DESCRIPTION OF THE PREFERRED EMBODIMENT

With reference now to the drawings, a preferred embodiment of the method of endodontic treatment is herein described. It should be noted that the articles “a”, “an”, and “the”, as used in this specification, include plural referents unless the content clearly dictates otherwise. The preferred embodiment of the method according to the invention utilizes two individual components. While not preferred, it should be noted that either of these components may be utilized individually to obtain an endodontic procedure that is still superior to the prior art.

The first component of the present invention is the use of a laser and matching dye to burn the residual soft tissue that remains in the canal after endodontic file therapy such that the residual tissue becomes carbonized. Burning the residual tissue within a root canal eliminates the major proprietor of secondary infection.

The second component of the present invention is the utilization of an anti-microbial agent when formulating endodontic sealers to provide a long-term anti-microbial effect within the obturated root canal. Loading an endodontic cement with a small portion of such agents can achieve long-term anti-microbial activity within the root canal. This protection provides additional protection in the event some necrotic tissue escapes the obliteration and burning of the laser. So, regardless of the leakage status of the restorations that repaired the pulp chamber and/or crown of the tooth, there is a means to disinfect any unwanted ingress that could eventually reach the root apex.

An embodiment of the present invention utilizes radiant energy to produce sufficient heat within the root canal that residual soft tissue is burned. The preferred radiant energy source is a diode laser, with the most preferred source of radiant energy is an 810 nm diode laser as it is a relatively inexpensive laser readily available for clinical settings. The preferred delivery is via a fiber optic cable or tip, wherein an unclad fiber optic end is inserted directly into the root canal and thereafter the laser is initiated to deliver the energy to the end of the fiber optic and into the canal. The preferred diameter of the fiber optic end is within the range of 100-300 microns, with the most preferred diameter is 200 microns. The preferred power output through a tip that measures between 100-400 microns in diameter is 0.2-5 watts, and the most preferred range is 0.5-3.5 watts. The preferred fiber optic cable is one with a high numerical aperture such that the beam is wide allowing light to be introduced onto the sidewalls of the canal.

An embodiment of the present invention utilizes dyes, stains, and pigments (collectively “dyes”) to stain any tissue remaining after extirpation. Each dye has its own absorption characteristics which are then matched to a laser with an output that closely matches at least one absorption ° max of any given dye. The preferred physical state of the dye is in liquid form and these dyes are usually combined with a solvent such as water, ethanol, acetone, propylene glycol, glycerin, and any other liquid solvent. The most preferred dye is indocyanine green as it is readily available, FDA approved, easily constituted into a liquid form, and has a λₘₐₓ at 810 nm, matching the preferred 810 nm diode laser. It is to be remembered that the most important characteristic of this component of the method of the invention is that the laser emission of radiant energy should have a wavelength A that is as close as possible to a λₘₐₓ of the dye to maximize the efficiency of energy absorption, ideally within a few nm (−1% λₘₐₓ) or up to a 10% variance. So, a laser which emits radiant energy between 730 and 890 nm would be considered a “match” for ICG, with those having a range of 801-819 (−1%) being preferred. Practitioners may choose a laser readily available to them and then dyes to match the laser output according to these guidelines. It should also be remembered that some dyes may have multiple λₘₐₓ characteristics and could work with lasers of multiple wavelengths.

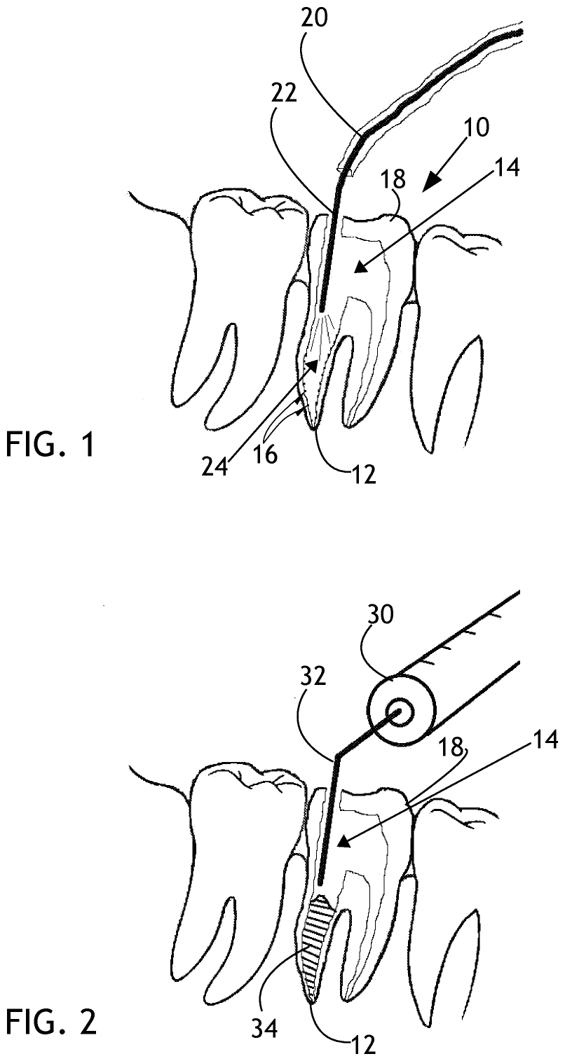

The preferred treatment method for this embodiment is to first gain access to the pulp chamber of the tooth (10) by conventional means such as with a high-speed handpiece, and then extirpate the necrotic root with endodontic files (as per the prior art). After these initial steps, ICG dye solution is introduced into the canal, allowing time for the solution to infiltrate residual soft tissues. After sufficient time has elapsed, the canal may be dried with paper points. Then, as shown in FIG. 1, an unclad end (22) of a fiber optic (20) connected to the radiant energy source, in this preferred case an 810 nm diode laser, is then inserted into the canal (14) and the 810 nm diode laser initiated. Upon absorption of emitted radiant energy (24), the ICG dye efficiently heats to such an extent that it begins to combust and burn residual necrotic tissue (16). The entire length of the canal (14) is treated from the root apex (12) to the coronal portion of the tooth (18) by slowly moving the tip in and out of the root canal (14) multiple times during the treatment. After laser treatment, the canal (14) may be treated with an antiseptic rinse, as would also be customary in the prior art, to kill pathogens and to rinse combusted tissues from the canal (14).

An embodiment of the present invention utilizes the salts of pyrithione, especially the zinc salt to provide long-term anti-microbial activity when added to restorative cement. The preferred range of loading zinc pyrithione within an inorganic cement is about 0.1%-1%; the most preferred range is 0.2%-0.5% by weight. An example of a light cured resin sealer/cement formula could be:
60% barium boroaluminasilicate powder 1.5 micron (radiopaque glass filler)
5%—hydroxyethyl methacrylate monomer
10%—diurethane dimethacrylate monomer
23%—Bis-GMA monomer
0.8%—dimethylaminoethyl methacrylate (initiator)
0.7% —camphorquinone (photo-initiator)
0.5%—zinc pyrithione
   Other cements, restorative resins, and materials may be manufactured utilizing the guidance provided herein with known compositions and techniques. The simple addition of an anti-microbial such as zinc pyrithione, especially in the quantities described, would not disrupt the physical properties of most known restorative cements and resins that are necessary to form lasting restorations.

The preferred treatment method for applying the anti-microbial root canal cement (FIG. 2) is to insert the mixed hydraulic cement or sealer directly into the canal (14) by means of a syringe (30) with a fine tip (32), a lentulo spiral, and/or any other endodontic delivery method. Thereafter, the cement (34) is allowed to cure into a solid form. The anti-microbial imbedded within the cured cement is designed to provide a long-term bactericidal and bacteriostatic effect within the canal in case the undesired ingress of microbes caused by leakage unfortunately occurs.

Although the present invention has been described with reference to preferred embodiments, numerous modifications and variations can be made and still the result will come within the scope of the invention. No limitation with respect to the specific embodiments disclosed herein is intended or should be inferred.

### INDUSTRIAL APPLICABILITY

The present invention has industrial applicability as its methods and materials are used in dentistry.

What is claimed is:

1. A method of endodontic therapy comprising: a first step of opening a pathway to gain access to the pulp chamber; a second step of extirpating infected tissue within the pulp chamber with endodontic files, thereby forming a canal; a third step of applying dye to the canal such that any residual infected tissue is stained; and, a fourth step of applying radiant energy from an energy source into the canal
with sufficient intensity such that stained tissue is burned; wherein the dye and energy source are selected due to the dye having a λₘₐₓ matched to a wavelength of radiant energy emitted from the radiant energy source.

1. The method of claim 1, the dye being indocyanine green.

2. The method of claim 2, the radiant energy being provided by an 810 nm diode laser.

3. The method of claim 1, further comprising a fifth step of obturating the canal with a restorative material which has anti-microbial properties.

4. The method of claim 4, the restorative material further comprising zinc pyrithione.

5. A restorative endodontic sealer comprising an anti-microbial constituent.

6. The restorative endodontic sealer of claim 6, the anti-microbial constituent being zinc pyrithione.

8. A method of endodontic therapy comprising: a first step of opening a pathway to gain access to the pulp chamber; a second step of extirpating infected tissue within the pulp chamber with endodontic files, thereby forming a canal; and a third step of obturating the canal with a restorative material which has anti
microbial properties; wherein the anti-microbial nature of the restorative material inhibits infection from residual infected tissue.

9. The method of claim 8, the restorative material comprising zinc pyrithione.
